(19) United States
(12) Patent Application Publication
(10) Pub. No.: US 2026/0241206 A1
(43) Pub. Date: Aug. 20, 2026

(54) RADIATION THERAPY SYSTEM, MOTION TRACKING DEVICE, AND MOTION TRACKING METHOD

(71) Applicants: Hitachi, Ltd., Tokyo (JP); NATIONAL UNIVERSITY CORPORATION HOKKAIDO UNIVERSITY, Sapporo-shi, Hokkaido (JP)

(72) Inventors: Ling Fung CHEUNG, Tokyo (JP); Takaaki FUJII, Tokyo (JP); Shinichiro FUJITAKA, Tokyo (JP); Naoki MIYAMOTO, Sapporo-shi, Hokkaido (JP); Kikuo UMEGAKI, Sapporo-shi, Hokkaido (JP); Koichi MIYAZAKI, Sapporo-shi, Hokkaido (JP)

(21) Appl. No.: 18/995,200

(22) PCT Filed: Mar. 10, 2023
(86) PCT No.: PCT/JP2023/009252

§ 371 Date: Jan. 16, 2025

(30) Foreign Application Priority Data
Aug. 30, 2022 (JP) 2022-137185

(51) Int. Cl. A61N 5/10; G06T 7/20; G16H 30/40

(52) U.S. Cl. CPC A61N 5/1037; G06T 7/20; G16H 30/40; G06T 2207/30024

(57) ABSTRACT

A motion tracking device includes: a medical image extractor which extracts motion information from medical images of a patient, the motion information being information regarding motions of a target, a tissue around the target, and a surrogate that is a characteristic site in a specific region; a motion model generator which constructs a motion model representing a correlation between the motions of the target, the tissue, and the surrogate based on the motion information; a motion detector which measures a motion of the surrogate during therapy in which the patient is irradiated with therapy radiation; a motion estimator which estimates current or future positions of the target and the tissue based on the motion model and the motion of the surrogate; and a motion model corrector which corrects the estimated positions of the target and the tissue during the therapy according to a predetermined correction protocol.

### TECHNICAL FIELD

The present disclosure relates to radiation therapy.

### BACKGROUND ART

In radiation therapy, a therapy beam of ionizing radiation (hereinafter, also simply referred to as radiation) such as high-energy electromagnetic waves and particles is irradiated toward a target such as a tumor to destroy cancer cells. Therefore, it is important to accurately irradiate the target with the therapy beam. However, as the patient breathes, the body moves and the position of the target is changed, which may affect the therapy beam irradiation accuracy. The decrease in irradiation accuracy causes overdosage to healthy body tissues or underdosage to the target. Therefore, in order to reduce errors and uncertainties in radiation therapy, it is important to manage the motions of the body.

Commonly used motion management methods include breath holding (breath holding technique), gating, motion tracking (moving object tracking technique), and the like. The breath holding technique has limited applicability to patients because not all the patients are not able to hold their breath during treatment. In gating, the therapy beam is switched on and off in accordance with the motion of the target accompanying respiration. Gating can be used in conjunction with the moving object tracking technique to track the motion of the target in real time during therapy.

As one method of tracking the motion of the target in real time using the moving object tracking technique, an external marker may be placed on the body surface of the patient, and the motion of the target may be tracked using the external marker. The external marker and the motion of the target are associated with each other by a correlation model. However, the motion of the external marker is not always invariably correlated with the motion of the target. Therefore, the correlation model needs to be updated frequently. In addition, it is often that the external marker results in a decrease in target tracking accuracy.

As another method of tracking the motion of the target in real time using the moving object tracking technique, an internal index marker may be used by surgically implanting a metal substance into the body. The internal index marker makes it possible to more accurately track the motion of the target. However, in order to obtain a high-contrast image, the tracking of the moving object based on the internal index marker requires implanting a high-density metal substance into the body, which has the following disadvantages. First, the internal indicator marker is invasive and poses a potential risk (such as pneumothorax) to the patient. Second, the internal indicator marker may move inside the body of the patient, affecting the tracking accuracy. Third, the internal indicator marker is likely to cause artifacts in medical images.

On the other hand, a tracking technique for accurately tracking the motion of the target in a non-invasive manner without using a marker has been proposed. Typically, the position of the target is specified using fluoroscopic digital X-ray images (digital radiography: DR) taken during therapy.

PTL 1 and PTL 2 disclose a method of tracking a target without using a marker by collating a digitally reconstructed radiograph (DRR) with DR.

Both PTL 1 and PTL 2 also mention the use of a surrogate, which is a characteristic site that is more easily recognized as a landmark on an image than the target on DR. PTL 1 describes a method for obtaining a movement of a target from a movement of a surrogate using a conversion parameter. PTL 2 describes a method of creating a correlation between a surrogate and a target using machine learning and using the correlation.

In addition, PTL 3 discloses a technique using a four-dimensional CT (4DCT) image. First, a mathematical model is constructed based on the 4DCT image. The mathematical model is then used together with DRRs and DRs generated from the 4DCT to specify the position of the target. In this approach, the position of the target is specified in almost real time. The dose distribution can be obtained from the mathematical model.

In the technique disclosed in NPL 1, a motion model is constructed using both a 4DCT image and an MRI image. By using medical images in a plurality of modalities, more detailed information can be incorporated into the motion model. By inputting a motion of a surrogate to the motion model, a volume image can be synthesized in almost real time. Dose calculations can be performed based on the synthesized volume image.

### CITATION LIST

### Patent Literature

PTL 1: JP 2017-144000 A
PTL 2: JP 2021-166730 A
PTL 3: JP 5134957 B2

### Non Patent Literature

NPL 1: Noemi Garau, Riccardo Via, Giorgia Meschini, Danny Lee, Paul Keal, Marco Riboldi, Guido Baroni and Chiara Paganelli, “A ROI-based global motion model established on 4DCT and 2D cine-MRI data for MRI-guidance in radiation therapy”, Physics in Medicine and Biology, vol. 64, no. 4, pp 45002, 2019.

### SUMMARY OF INVENTION

### Technical Problem

In general, a markerless tracking method relies on DRs to directly track a motion of a tumor. However, for a tumor at a particular body site such as the liver or the pancreas, it is difficult to obtain a good-contrast image necessary for directly finding the tumor from an image such as DR. This limits the availability of markerless tracking.

The techniques of PTL 1 and PTL 2 aim to solve this problem by using a surrogate. However, the conversion parameter used in PTL 1 changes during therapy depending on the relationship between the tumor and the surrogate in the DRR image, which may decrease the accuracy in specifying the position of the tumor. In addition, the machine learning used in PTL 2 generally requires extensive training data, and the obtained correlation between the surrogate and the target is sensitive to the used training data set.

In addition, in particle beam therapy (PBT), when an anatomical change occurs in an area of the body through which a particle beam passes, the range of the particle beam in the body changes. Therefore, the motion of the tissue around the tumor may greatly affect the range irradiated with the particle beam. However, in the techniques of PTL 1 and PTL 2, it is not possible to obtain information regarding the motion of the tissue around the tumor, which is important in obtaining high accuracy.

In both the techniques of PTL 3 and NPL 1, a mathematical motion model constructed from medical tomographic images is used to estimate the motion of the target in almost real time. These methods make it possible to obtain information about the motion of the tissue surrounding the tumor. However, the motion model is created based on the correlation between the tumor and the surrogate established at the time when the medical tomographic images are captured. Meanwhile, the correlation between the tumor and the surrogate may change even during therapy. Therefore, in the motion model based on the correlation at the time when the medical tomographic images are captured, there is a possibility that an error may occur in tracking the moving object in real time.

Furthermore, all the methods described above may have errors due to latency. The latency is a time difference between a time when a DR image is taken and a time when a therapy beam is delivered to an estimated position of a tumor. If this latency is too long, tracking accuracy may significantly decrease, especially when the tumor is moving rapidly.

One object of the present disclosure is to provide a technology for improving accuracy in irradiating a target with therapy radiation.

### Solution to Problem

A radiation therapy system according to one aspect of the present disclosure includes: a motion tracking device including: a medical image extractor which extracts motion information from medical images of a patient, the motion information being information regarding motions of a target to be irradiated with therapy radiation, a tissue around the target, and a surrogate that is a characteristic site in a specific region of the patient; a motion model generator which constructs a motion model representing a correlation between the motions of the target, the tissue, and the surrogate based on the motion information; a motion detector which measures a motion of the surrogate during therapy in which the patient is irradiated with the therapy radiation; a motion estimator which estimates current or future positions of the target and the tissue based on the motion model and the motion of the surrogate; a motion model corrector which corrects the estimated positions of the target and the tissue during the therapy according to a predetermined correction protocol; and a therapy controller which is configured to deliver the therapy radiation to the target of the patient based on the estimated current or future positions of the target and the tissue.

### Advantageous Effects of Invention

According to one aspect of the present disclosure, the accuracy in irradiating the target with therapy radiation can be improved.

### BRIEF DESCRIPTION OF DRAWINGS

FIG. 1 is a conceptual diagram showing an example of a particle beam therapy system.

FIG. 2 is a diagram showing how a patient is imaged.

FIG. 3 is a schematic flowchart of overall processing executed by the particle beam therapy system.

FIG. 4 is a flowchart of processing of constructing a motion model.

FIG. 5 is a flowchart of processing of obtaining an estimation result using the motion model.

### DESCRIPTION OF EMBODIMENTS

Hereinafter, an embodiment of the present invention will be described with reference to the drawings.

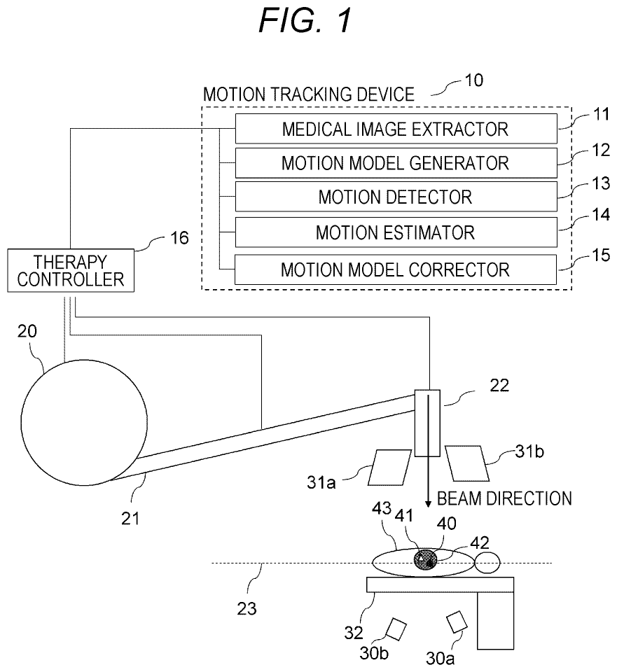

FIG. 1 is a conceptual diagram showing an example of a particle beam therapy system according to the present embodiment.

The particle beam therapy system is a system that irradiates a target 40 with a particle therapy beam, the target 40 being a tumor of a patient 43 placed on a therapy platform 32. The particle beam therapy system includes a motion tracking device 10, a therapy controller 16, an accelerator 20, a beam delivery system 21, a gantry 22, a pair of X-ray sources 30, and an X-ray image detector 31.

The motion tracking device 10 is a device that tracks motions of a target 40, its surrounding tissue, and a surrogate 41 in a region of interest (ROI) 42 including the target 40, estimates their positions at a future time, and outputs an estimation result. The surrogate 41 is a characteristic site that is relatively easy to identify on images.

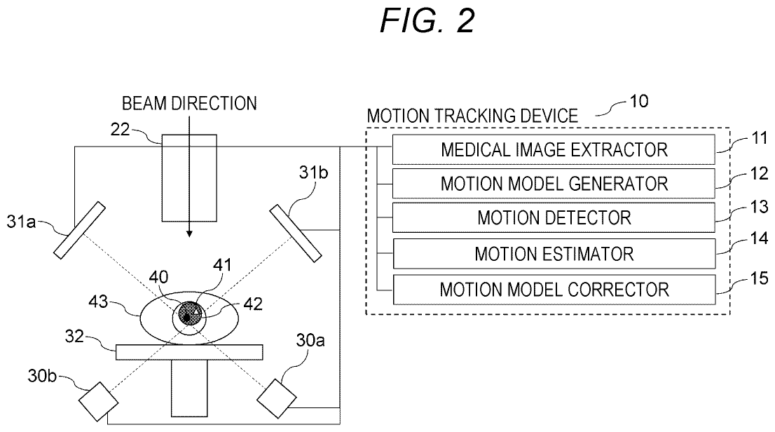

FIG. 2 is a diagram showing how a patient is imaged. FIG. 2 shows a therapy platform 32 viewed from the direction of a rotation axis 23 shown in FIG. 1.

X-ray sources 30a and 30b emit X-rays that pass through the body of the patient 43 to capture images toward the ROI 42 of the patient. X-ray image detectors 31a and 31b detect X-rays that have been emitted from the X-ray sources 30a and 30b and passed through the ROI 42 of the patient 43 to capture images, and creates digital radiographic images. The digital radiographic images are sent to the motion tracking device 10 and used in a motion tracking process.

The therapy controller 16 controls the accelerator 20, the beam delivery system 21, and the gantry 22 to irradiate the target 40 with the therapy beam based on a therapy plan created in advance and current or future positions of the target and the tissue, which are obtained as an estimation result by the motion tracking device 10.

The accelerator 20 accelerates charged particles until the charged particles have appropriate energy, and outputs the accelerated charged particles as therapy radiation. The therapy radiation output from the accelerator 20 is delivered to the gantry 22 by the beam delivery system 21. The gantry 22 irradiates the patient 43 with the therapy radiation delivered by the beam delivery system 21 as a therapy beam. The gantry 22 can rotate around the patient 43 about the rotation axis 23 to irradiate a tumor of the patient 43, which is the target 40, with the therapy beam from various angles.

The motion tracking device 10 is a computer that is operated by a processor executing a software program, and the processor executes the software program to implement a medical image extractor 11, a motion model generator 12, a motion detector 13, a motion estimator 14, and a motion model corrector 15 shown in FIGS. 1 and 2.

The medical image extractor 11 acquires medical images, which are digital radiographic images of the patient 43, from a medical image server (not shown) in advance before therapy during which the patient 43 is actually irradiated with therapy radiation. Here, the medical images refer to computed tomography (CT) images, magnetic resonance imaging (MRI) images, and cone beam CT (CBCT) images. The medical image extractor 11 extracts, from the medical images of the patient 43, motion information that is information regarding motions of the target 40 to be irradiated with therapy radiation, the tissue around the target 40, and the surrogate 41 that is a characteristic site in the region-of-interest 42 of the patient 43.

Based on the motion information extracted by the medical image extractor 11, the motion model generator 12 constructs a motion model representing a correlation between the motions of the target 40, the tissue, and the surrogate 41 in the region-of-interest 42.

The motion detector 13 measures a motion of the surrogate 41 during actual therapy from medical images obtained by the X-ray sources 30a and 30b and the X-ray image detectors 31a and 31b.

Based on the motion model generated by the motion model generator 12 and the motion of the surrogate 41 measured by the motion detector 13, the motion estimator 14 estimates current or future positions of the target 40 and its surrounding tissue.

The motion model corrector 15 corrects the positions of the target 40 and its surrounding tissue estimated by the motion estimator 14 during the therapy according to a predetermined correction protocol. An estimation result indicating the corrected positions of the target 40 and its surrounding tissue is provided to the therapy controller 16.

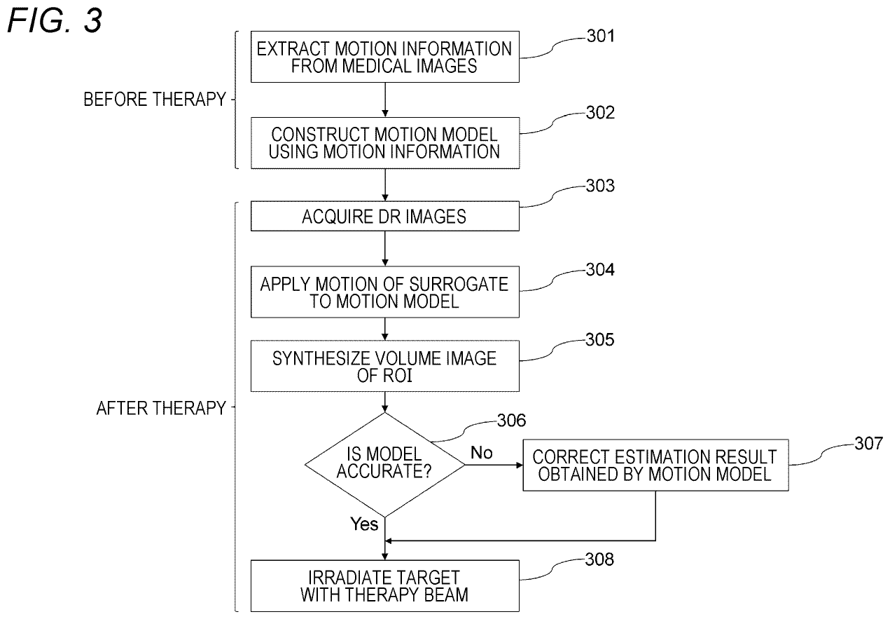

FIG. 3 is a schematic flowchart of overall processing executed by the particle beam therapy system.

In step 301, the medical image extractor 11 extracts motion information of the target 40, the tissue in the ROI 42, and the surrogate 41 from medical images. In step 302, the motion model generator 12 constructs a motion model using the motion information extracted in step 301. Steps 301 and 302 are processes performed before therapy. Step 303 and subsequent steps are processes performed during therapy. In step 303, the motion detector 13 acquires DR images and motion information on a position, a velocity, and an acceleration of the surrogate 41 during therapy. In step 304, the motion estimator 14 estimate positions of the target 40 and the tissue in the ROI 42 by applying the motion information of the surrogate 41 obtained in step 303 to the motion model constructed in step 302. In step 305, the motion estimator 14 synthesizes a volume image of the ROI 42. This volume image can be used for dose calculation.

In step 306, the motion model corrector 15 determines whether the accuracy of the motion model is high enough to be acceptable using the volume image. When the accuracy of the motion model is acceptable, the motion model corrector 15 provides the estimation result obtained in step 304 by the motion estimator 14 to the therapy controller 16. The therapy controller 16 irradiates the target 40 with a therapy beam based on the provided estimation result. When the accuracy of the motion model is not acceptable, the motion model corrector 15 corrects the estimation result obtained by the motion estimator 14 according to a predetermined correction protocol, and provides the corrected result to the therapy controller 16 in step 307.

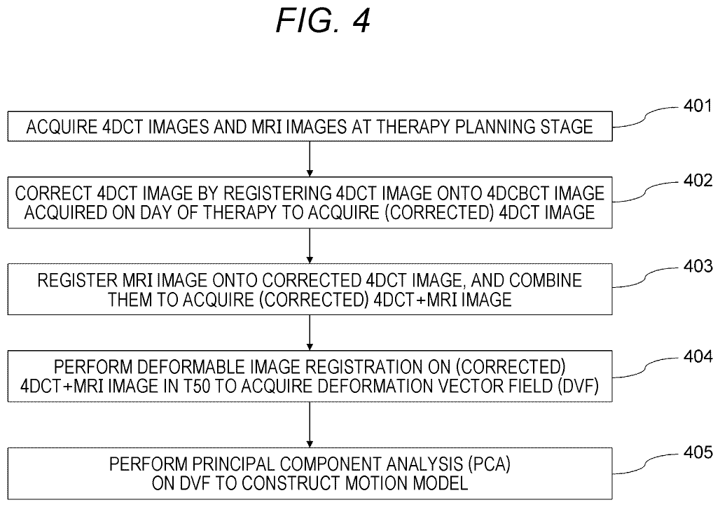

FIG. 4 is a flowchart of processing of constructing a motion model. This processing is processing corresponding to steps 301 and 302 in the entire processing shown in FIG. 3.

In step 401, the medical image extractor 11 acquires medical images of both modalities, a 4DCT image and an MRI image, of the patient 43 at a therapy planning stage. The 4DCT image only provides not important structural information for planning therapy, but also information about the respiratory motion of the patient 43. The MRI image improves tumor contours, and provides supplemental structural information for detecting body tissues for excellent soft tissue contrast.

On the day of therapy, there is usually an inter-fractional motion (a movement of a body tissue according to a change in body condition during the therapy period) different from that on the day on which the therapy plan is carried out, which may affect the accuracy of the therapy. A 4DCBCT image is taken to obtain structural information on the day of therapy.

In step 402, the medical image extractor 11 registers a previously acquired 4DCT image onto the 4DCBCT image taken on the day of therapy, considering the change in inter-fractional motion, to obtain a (corrected) 4DCT image.

In step 403, the medical image extractor 11 registers a previously acquired MRI image to the (corrected) 4DCT image to generate a (corrected) 4DCT+MRI image. The (corrected) 4DCT+MRI image includes not only information from both the 4DCT and the MRI but also correction using the 4DCBCT image indicating the condition of the patient 43 on the day of therapy.

In step 404, the medical image extractor 11 performs deformable image registration (DIR) on the (corrected) 4DCT+MRI image, using an image in a full expiration phase (T50) as an image in a reference phase. That is, all images at respiratory phases other than T50 are subjected to deformable image registration to match the respiratory phase of T50. This registration produces a deformation vector field (DVF) that quantitatively describes how various parts of the body move during breathing. The deformation vector of each voxel in the deformation vector field represents how the voxel moves relative to the reference respiratory phase in each phase.

Since the number of voxels in an image is enormous, it is not realistic to set up motion equations individually for all the voxels. Therefore, in step 405, the motion model generator 12 constructs a motion model that approximately describes motions of the target and the tissue in the ROI, using principal component analysis (PCA).

First, the motion model generator 12 extracts displacement vector dⱼ of each voxel of the ROI for different respiratory phases from the DVF, which is a DIR result, as shown in Equation (1).

$$\begin{matrix}[ Mathematical formula 1 ] & \\ {d}_{j} = {[ {u}_{1 , j} , {u}_{2 , j} , ... , {u}_{m , j} , ... , {u}_{M , j} ]}^{T} & ( 1 )\end{matrix}$$

Here, u_{m,j} is a displacement vector of voxel m at time j (0<j<J, J is the number of time frames in the respiratory phase). M is the total number of voxels.

Next, the motion model generator 12 constructs a matrix D shown in Equation (2).

$$\begin{matrix}[ Mathematical formula 2 ] & \\ D = [ {d}_{1} - \overline{d} , ... , {d}_{j} - \overline{d} , ... , {d}_{J} - \overline{d} ] & ( 2 )\end{matrix}$$

where d̅ is a mean vector

$$\frac{1}{J} {\sum }_{j = 1}^{J} {d}_{j}$$

of a motion.

DD^{T} represents a covariance matrix of data corresponding to a motion. Therefore, according to the principles of principal component analysis, an eigenvector of the DD^{T} can be used with the largest eigenvalue (principal vector) to represent a principal component of the motion. Since there are millions of voxels in each image (M □ 10⁶), it is not realistic to directly calculate the covariance matrix DD^{T} of eigenvectors where D is an M×J matrix. Therefore, an eigenvalue λ satisfying D^{T}DX=λX is acquired with X as an eigenvector of the D^{T}D.

$$\begin{matrix}[ Mathematical formula 3 ] & \\ {DD}^{T} ( DX ) = D ( {D}^{T} DX ) = λ DX & ( 3 )\end{matrix}$$

Therefore, DX is an eigenvector of the covariance matrix DD^{T}, and λ is an eigenvalue of the DD^{T} and the D^{T}D. The matrix D^{T}D is a J×J matrix. The eigenvector X can be easily calculated.

After the principal vector is obtained, the approximation of the displacement vector d of any voxel at any time t can be expressed as the following Equation (4).

$$\begin{matrix}[ Mathematical formula 4 ] & \\ d ( t ) = {\sum }_{k = 1}^{K} {w}_{k} ( t ) {e}_{k} & ( 4 )\end{matrix}$$

Here, eₖ is a jₖ-th principal component vector. wₖ is an unknown weight parameter for eₖ. K is the total number of principal component vectors used. The weight parameter wₖ can be determined based on a motion of a surrogate, such as a diaphragm movement. The above Equation (4) can be divided into two independent equations, and can be expressed as Equations (5) and (6) in matrix notation.

$$\begin{matrix}[ Mathematical formula 5 ] & \\ s = {E}_{s} W & ( 5 )\end{matrix}$$

$$u = {E}_{u} W \qquad ( 6 )$$

Here, s is a K×1 matrix formed by displacement vectors of surrogates, and u is a J×1 matrix formed by displacement vectors of all the other voxels in the ROI. Eₛ is a K×K matrix formed principal by K component vectors of K surrogates. K surrogate coordinates are used such that Eₛ is reversible. Eᵤ is a J×K matrix formed by K principal vectors of J voxels. W is a K×1 matrix formed by weight parameters for each principal component vector. W can be removed as in Formula (7).

$$\begin{matrix}[ Mathematical formula 6 ] & \\ u ( t ) = {E}_{u} {E}_{s}^{- 1} s ( t ) & ( 7 )\end{matrix}$$

Therefore, a relationship that links motions of the surrogates to motions of all the other voxels in the ROI is obtained. Equation (7) shows that the displacement vectors of the other voxels at time t can be estimated by measuring the displacements of the surrogates at time t. From future motions s(t′) of the surrogates, future motions u(t′) of the other voxels in the ROI can be predicted.

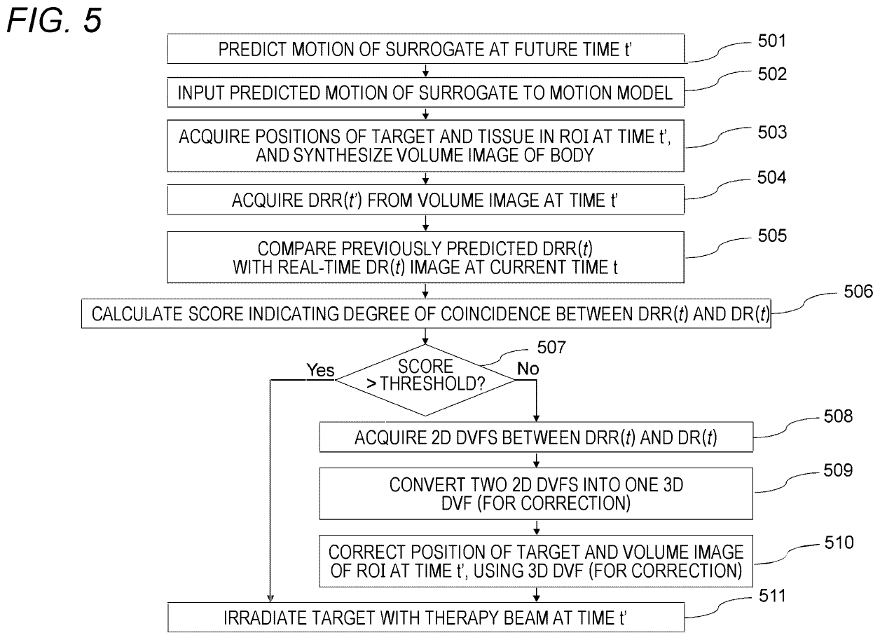

FIG. 5 is a flowchart of processing of obtaining an estimation result using the motion model. This processing is processing corresponding to steps 303 to 308 shown in FIG. 3.

In step 501, the motion detector 13 predicts a motion of the surrogate at future time t′. The motion of the surrogate at future time t′ can be calculated, for example, by utilizing the velocity and acceleration of the surrogate acquired at a past time of the motion of the surrogate at future time t′. In step 502, the motion estimator 14 inputs the predicted motion of the surrogate to the motion model.

In step 503, the motion estimator 14 can acquire positions of the target and the tissue at time t′. At this time, the volume image of the ROI at time t′ is also synthesized from the output of the motion model. A water equivalent thickness (WET) of a region through which a therapy beam passes can be calculated using a volume image of the ROI synthesized in almost real time. In step 504, the motion estimator 14 obtains a digitally reconstructed radiograph (DRR) image from the synthesized volume image at the future time.

In step 505, at current time t, the motion model corrector 15 compares the DRR image previously acquired for the current time t and a DR image at a current time point. In step 506, the motion model corrector 15 calculates a matching score indicating a degree of coincidence between the DRR (t) image and the DR (t) image.

In step 507, the motion model corrector 15 compares the calculated score with a predetermined threshold to determine whether the motion model needs to be corrected. When correction is necessary, the processing proceeds to step 508, and when correction is not necessary, the processing proceeds to step 511. In step 508, the motion model corrector 15 corrects the motion model using a difference between the DRR (t) image and the DR (t) image. With such a difference, two 2D DVFs for each imaging angle can be obtained.

In step 509, the motion model corrector 15 converts the two 2D DVFs into one 3D DVF, that is, a 3D DVF (for correction), via a conversion parameter between the imaging coordinate system and the treatment room coordinate system. In step 510, the motion model corrector 15 corrects the predicted position of the target in the ROI and the volume image of the ROI at future time t′ using the 3D DVF (for correction).

If the interval between time t′ and time t is short, more accurate correction can be performed. In a case where the interval between time t′ and time t is negligibly short, this can be regarded as real time. In step 511, the therapy controller 16 irradiates the target with therapeutic radiation according to the synthesized target position and volume image at time t′.

Although the embodiments of the present invention have been described above, the present invention is not limited to these embodiments, and these embodiments may be used in combination or some configurations may be changed within the scope of the technical idea of the present invention.

In addition, the above-described embodiments include the following items. However, the items included in the present embodiment are not limited only to those described below.

### (Item 1)

A radiation therapy system including:
a motion tracking device including: a medical image extractor which extracts motion information from medical images of a patient, the motion information being information regarding motions of a target to be irradiated with therapy radiation, a tissue around the target, and a surrogate that is a characteristic site in a specific region of the patient; a motion model generator which constructs a motion model representing a correlation between the motions of the target, the tissue, and the surrogate based on the motion information; a motion detector which measures a motion of the surrogate during therapy in which the patient is irradiated with the therapy radiation; a motion estimator which estimates current or future positions of the target and the tissue based on the motion model and the motion of the surrogate; and a motion model corrector which corrects the estimated positions of the target and the tissue during the therapy according to a predetermined correction protocol; and
a therapy controller which is configured to deliver the therapy radiation to the target of the patient based on the estimated current or future positions of the target and the tissue.

According to this item, it is possible to irradiate the target with the therapy radiation based on the future motion estimated and corrected during the therapy, thereby improving the accuracy with which the target is irradiated with the therapy radiation.

### (Item 2)

The radiation therapy system according to item 1, in which
the medical image extractor is configured to:
acquire medical images of the target, the tissue, and the surrogate in a plurality of modalities including a first modality and a second modality;
register the medical image of the first modality onto the medical image of the second modality;
combine the registered medical image of the first modality into the medical image of the second modality; and
extract the motion information from the combined medical images.

According to this item, it is possible to extract the motion information of the target, the tissue, and the surrogate from the medical image obtained by combining the images of the plurality of modalities, thereby constructing the motion model with high accuracy.

### (Item 3)

The radiation therapy system according to item 1, in which
the motion model is a model that indicates a deformation vector by a vector approximated by principal component analysis, the deformation vector representing a motion in each phase relative to a predetermined respiratory phase for each voxel in the specific region.

According to this item, it is possible to express complex motions of enormous voxels in a motion model that can be easily calculated by principal component analysis.

### (Item 4)

The radiation therapy system according to item 1, in which
the motion detector acquires a real-time medical image of the patient, and acquires a position, a velocity, and/or an acceleration of the surrogate from the real-time medical image.

### (Item 5)

The radiation therapy system according to item 1, in which
the motion estimator generates a vector field describing a relative position of each voxel at a reference time at each position in the specific region, and calculates positions of the target and the tissue at the reference time using the vector field.

### (Item 6)

The radiation therapy system according to item 5, in which
the motion estimator predicts a motion of the surrogate based on a position, a velocity, and/or an acceleration of the surrogate acquired, and applies the predicted motion of the surrogate to the motion model to predict motions of the target and the tissue.

### (Item 7)

The radiation therapy system according to item 6, in which
the motion estimator synthesizes a volume image describing positions of the target and the tissue in the specific region based on the prediction of the motions of the target and the tissue.

### (Item 8)

The radiation therapy system according to item 1, in which
the correction protocol specifies an accuracy of the motion model by comparing a synthesized medical image based on the positions of the target and the tissue estimated by the motion estimator with a real-time medical image acquired by the medical image extractor, and corrects the positions of the target and the tissue estimated by the motion estimator based on a difference between the synthesized medical image and the real-time medical image when the accuracy is lower than or equal to a predetermined threshold.

### REFERENCE SIGNS LIST

10 motion tracking device
11 medical image extractor
12 motion model generator
13 motion detector
14 motion estimator
15 motion model corrector
16 therapy controller
20 accelerator
21 beam delivery system
22 gantry
23 rotation axis
30 x-ray source
31 X-ray image detector
32 therapy platform
40 target
41 surrogate
42 region of interest (ROI)
43 patient

1. A radiation therapy system comprising:
a motion tracking device including:
a medical image extractor which extracts motion information from medical images of a patient, the motion information being information regarding motions of a target to be irradiated with therapy radiation, a tissue around the target, and a surrogate that is a characteristic site in a specific region of the patient;
a motion model generator which constructs a motion model representing a correlation between the motions of the target, the tissue, and the surrogate based on the motion information; a motion detector which measures a motion of the surrogate during therapy in which the patient is irradiated with the therapy radiation;
a motion estimator which estimates current or future positions of the target and the tissue based on the motion model and the motion of the surrogate; and
a motion model corrector which corrects the estimated positions of the target and the tissue during the therapy according to a predetermined correction protocol, and
a therapy controller which is configured to deliver the therapy radiation to the target of the patient based on the estimated current or future positions of the target and the tissue.

2. The radiation therapy system according to claim 1, wherein
the medical image extractor is configured to:
acquire medical images of the target, the tissue, and the surrogate in a plurality of modalities including a first modality and a second modality;
register the medical image of the first modality onto the medical image of the second modality, and
combine the registered medical image of the first modality into the medical image of the second modality; and
extract the motion information from the combined medical images.

3. The radiation therapy system according to claim 1, wherein the motion model is a model that indicates a deformation vector by a vector approximated by principal component analysis, the deformation vector representing a motion in each phase relative to a predetermined respiratory phase for each voxel in the specific region.

4. The radiation therapy system according to claim 1, wherein the motion detector acquires a real-time medical image of the patient, and acquires a position, a velocity, and/or an acceleration of the surrogate from the real-time medical image.

5. The radiation therapy system according to claim 1, wherein the motion estimator generates a vector field describing a relative position of each voxel at a reference time at each position in the specific region, and calculates positions of the target and the tissue at the reference time using the vector field.

6. The radiation therapy system according to claim 5, wherein the motion estimator predicts a motion of the surrogate based on a position, a velocity, and/or an acceleration of the surrogate acquired, and applies the predicted motion of the surrogate to the motion model to predict motions of the target and the tissue.

7. The radiation therapy system according to claim 6, wherein the motion estimator synthesizes a volume image describing positions of the target and the tissue in the specific region based on the prediction of the motions of the target and the tissue.

8. The radiation therapy system according to claim 1, wherein the correction protocol specifies an accuracy of the motion model by comparing a synthesized medical image based on the positions of the target and the tissue estimated by the motion estimator with a real-time medical image acquired by the medical image extractor, and corrects the positions of the target and the tissue estimated by the motion estimator based on a difference between the synthesized medical image and the real-time medical image when the accuracy is lower than or equal to a predetermined threshold.

9. A motion tracking device comprising:
a medical image extractor which extracts motion information from medical images of a patient, the motion information being information regarding motions of a target to be irradiated with therapy radiation, a tissue around the target, and a surrogate that is a characteristic site in a specific region of the patient;
a motion model generator which constructs a motion model representing a correlation between the motions of the target, the tissue, and the surrogate based on the motion information;
a motion detector which measures a motion of the surrogate during therapy in which the patient is irradiated with the therapy radiation;
a motion estimator which estimates current or future positions of the target and the tissue based on the motion model and the motion of the surrogate; and
a motion model corrector which corrects the estimated positions of the target and the tissue during the therapy according to a predetermined correction protocol.

10. A motion tracking method comprising:
extracting motion information from medical images of a patient, the motion information being information regarding motions of a target to be irradiated with therapy radiation, a tissue around the target, and a surrogate that is a characteristic site in a specific region of the patient;
constructing a motion model representing a correlation between the motions of the target, the tissue, and the surrogate based on the motion information;
measuring a motion of the surrogate during therapy in which the patient is irradiated with the therapy radiation;
estimating current or future positions of the target and the tissue based on the motion model and the motion of the surrogate; and
correcting the estimated positions of the target and the tissue during the therapy according to a predetermined correction protocol.
